(19) United States
(12) Patent Application Publication
(10) Pub. No.: US 2008/0177255 A1
(43) Pub. Date: Jul. 24, 2008

(54) THERAPEUTIC COIFFURING INSTRUMENT

(75) Inventor: Allen J. BERNARDINI, Morris, CT (US)

Correspondence Address: LOWE HAUPTMAN HAM & BERNER, LLP, 1700 DIAGONAL ROAD, SUITE 300, ALEXANDRIA, VA, 22314, US

(73) Assignee: R3 CONNECTOR SYSTEMS, Chino, CA (US)

(21) Appl. No.: 11/625,398

(22) Filed: Jan. 22, 2007

(51) Int. Cl. A61B 18/18; A61B 18/00; A45D 7/00

(52) U.S. Cl. 606/3; 606/16; 132/200

(57) ABSTRACT

A therapeutic coiffuring instrument has a light source disposed with the therapeutic coiffuring instrument to produce light selected to produce therapeutic effects to scalp and hair. Elongate members forming part of the instrument are configured to contact hair and scalp and to conduct the light from the source of light into contact with the hair and scalp.

### BACKGROUND OF THE INVENTION

1. Field of the Invention

The present invention relates generally to therapeutic coiffuring instrument which can be used to promote healthy hair, scalp, body and sheen, and more specifically to a therapeutic coiffuring instrument which is equipped with a light source configured to promote the aforementioned healthy hair, scalp, body and sheen.

2. Description of the Related Art

It is known that LED (Light Emitting diodes) have curative capability when configured to various wavelengths. Articles have been published indicating that the use of LEDs can be used for healing muscle or joint pain, for faster healing of cuts and other wounds, to repair sun damage and other deleterious effects on human skin, to whiten teeth, etc.

However, the above efforts have overlooked the possibility of using this type of treatment in connection with balding or other hair/hair loss problems and the like in both men and women.

### SUMMARY OF THE INVENTION

Embodiments of the invention are directed to arrangements wherein tuned wavelengths of light are applied directly to the scalp and hair via a coiffuring instrument with the aim of producing a healthier scalp and more lustrous hair.

The embodiments of the invention are directed to a coiffuring instrument such as a hairbrush or a comb which has possible ancillary health effects, and more specifically is directed to a comb or hairbrush having the teeth or bristles capable of transmitting either by “end illumination” or “side emitted illumination” methods, light whose wavelength can be selected or varied for cosmetic and therapeutic purposes.

As noted above, it is known that “light”—particularly light of specific wavelengths, has therapeutic or healing capabilities. Equipment exists today to “light bathe” injured or infected tissue to help promote healing and to rejuvenate epidermal layers of skin. This equipment is in use today by doctors, dentists, cosmeticians and the like. In addition to the beneficial effects the light sources which are used in the embodiments of the invention are low power LED's which have the distinct advantage of long life (25,000 hours plus), very low power consumption (Milliwatts) and color variability (Light Wave Length).

Many aids exist on the market today in the form of creams, gels, lotions, potions and gadgets to promote healthy hair and scalp, by presumably assisting to provide body, flexibility and luster to hair—human and animal. Many of these same aids are sold to promote scalp vitality and hair follicle health.

Utilizing a coiffuring instrument such as a comb or brush, whose teeth or bristles can provide low level light having a specific wave length or wave lengths directly to the hair and scalp while combing or brushing, can assist in the process of making one's scalp and hair—healthier, shinier, and fuller.

The LED light source can be powered by rechargeable/replaceable batteries—much like today's cordless toothbrushes or directly from an AC voltage source with the proper voltage adapter. The batteries and the light source can be located in the handle of the device which can be detachable for either battery or head replacement or for sanitary purposes or charging, etc.

Charging can be carried out by plugging directly into a charger or by inductive means in a battery charging holder.

The optical fibers, which transmit the light, can be glass or plastic. The optical fibers can be imbedded into the comb teeth or brush bristles during the fabrication of the head or they can be the actual teeth or bristles with capability of light transmission.

The LED's can be selected for light wavelength and intensity or they can be “tuned” by a simple electronic control in the handle.

The LED's can be configured to facilitate light transfer to any comb or brush array and shape and to also optimize production techniques.

Removal of the comb or brush head from the handle will provide a wide cone of light, which can be used in emergencies as a flashlight. In addition, as LED's can be selected or tuned to replicate daylight (5600 K), the handle can be used as a very effective cosmetic application light.

The light conducting fibers can also be configured for light to be transmitted exclusively from the ends, or from the ends and sides, or exclusively from the sides to obviate any concern of the light from the ends of the teeth or bristles producing a detrimental optical effect which might occur if a user were to look directly at the ends of the teeth.

### BRIEF DESCRIPTION OF THE DRAWINGS

The present invention is illustrated by way of example, and not by limitation, in the figures of the accompanying drawings, wherein elements having the same reference numeral designations represent like elements throughout and wherein:

FIG. 1 is a schematic diagram depicting the basic arrangement of an embodiment of the invention;

FIG. 2 is a schematic diagram depicting the manner in which light conducting elements can be bent in a manner which facilitates the formation of a therapeutic coiffuring instrument according to the present invention.

FIGS. 3 and 4 are respectively perspective views illustrating possible embodiments of the therapeutic coiffuring instrument to which the invention is directed.

FIG. 5 is a perspective view of showing a handle of a brush or comb such as illustrated in FIGS. 3 and 4 removed and from the body of the therapeutic coiffuring instrument and disposed in a manner which permits its use as a cosmetic light or the like.

FIGS. 6 and 7 are perspective views showing teeth/bristle arrangements which can be used conduct light to hair and scalp.

FIG. 8 is a schematic illustration of a circular bundle of light conducting fibers being supplied with light from a light source and then drawn out into a functional curved fan shape.

FIGS. 9 and 10 illustrate arrangements wherein light conducting fibers are enclosed in shaped bodies in accordance with embodiments of the invention.

FIG. 11 is a schematic drawing depicting the manner in which mirrors can be used to redirect light in lieu of the use of light conducting fibers.

### DETAILED DESCRIPTION OF THE EXEMPLARY EMBODIMENTS

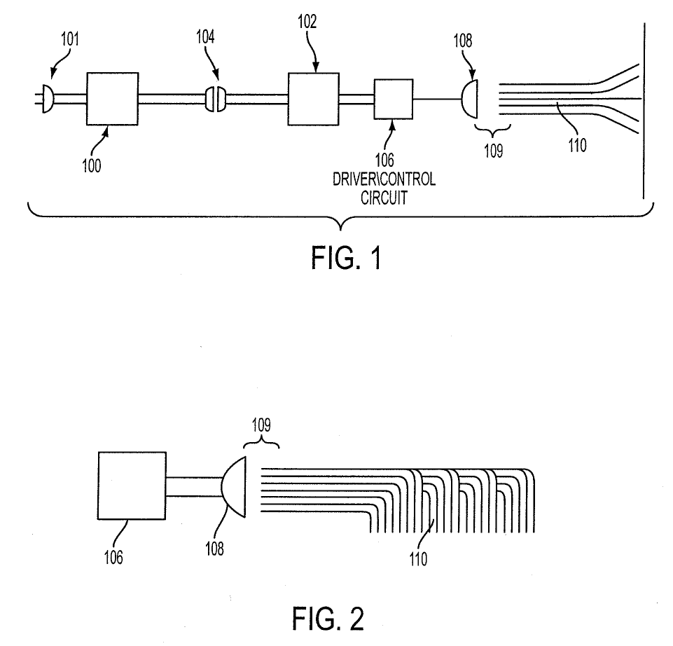

FIG. 1 illustrates in schematic form, a circuit/fiber arrangement which is used in at least one embodiment of the invention. In this arrangement a power supply 100 in the form of an AC/DC converter is configured to be connected with household AC outlet via a plug 101. The converter 100 is configured to be selectively connected with a rechargeable battery 102 via a connector arrangement 104.

A driver/control circuit 106 is operatively connected with the battery 102 to receive DC current therefrom and further configured to illuminate a light source 108. In this embodiment the light source 108 comprises a plurality of LED (Light Emitting Diodes). This light source 108 is optically coupled via optical coupling 109 with a plurality of light conducting fibers 110 which are arranged to conduct the light from the light source to a point of delivery where the light from the LED is emitted. Inasmuch as the embodiments of the invention are directed to a therapeutic coiffuring instrument, the point of delivery of the LED light is intended to be the scalp and/or hair of a user.

The optical coupling 109, in the above instance, can take the form of any suitable light transmission device which may or may not have a lens effect and which modifies the light emitted from the LED and directs it toward the ends of the light conducting fibers 110 in a manner which tends to maximize the amount of light which is transmitted via the fibers 110. In its simplest form, however, the optical coupling can simply take the form of an air-gap.

The driver/control circuit 106 is arranged to permit either or both of intensity and wavelength (color) of the light produced by the LED to be adjusted in accordance with the intended use.

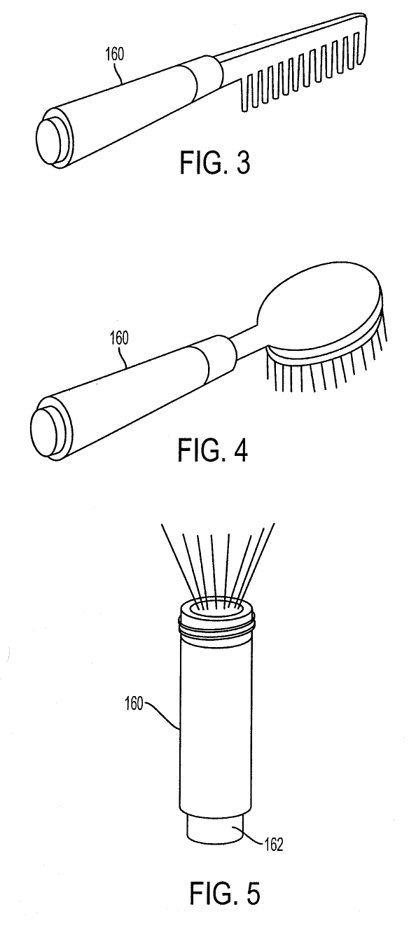

FIGS. 2 and 8 illustrate how the bundles of light transmitting fibers 110 can be configured in accordance with the need to direct light to the scalp/hair and thus be useful in arrangements such as illustrated in FIGS. 3 and 4. The bristles/teeth of the instruments shown in FIGS. 3 and 4, or the fibers illustrated in FIGS. 2 and 8 can be configured so that all of the light which is transmitted through the light transmitting elements (e.g. bristles, teeth or fibers) can be delivered through the ends or tips of the same. Alternatively, the tips be treated to have an opaque/reflective characteristic and the elements be shaped or otherwise configured to cause the light to emitted through the sides thereof. A combination of these arrangements such as in a 50/50 alternating arrangement, is also possible without departing from the scope of the invention.

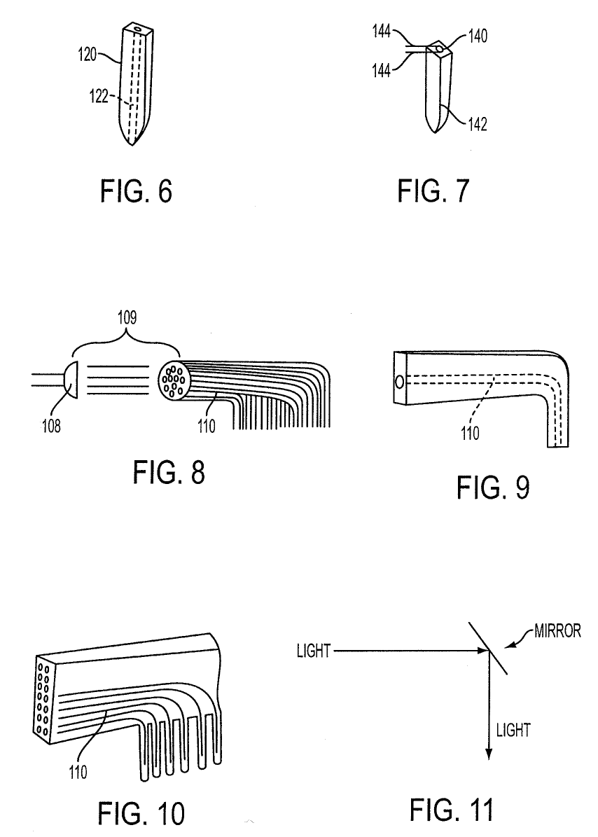

FIG. 6 shows a tooth/bristle 120 which is provided with a hollow portion or passage 122 through which light can be transmitted (viz., through the air contained therein) or have a light transmitting fiber disposed therein. FIGS. 9 and 10 show arrangements wherein fibers 110 are embedded in the shaped portion of a coiffuring instrument.

FIG. 7 shows another embodiment wherein an organic LED (viz., OLED) 140 is disposed on the top of a light transmitting element 142 which also servers a tooth/bristle of a coiffuring instrument and thus provided a patch for the therapeutic light to be delivered to the scalp/hair of a user. The numeral 144 denotes the lead wires which are used to supply current to the OLED 140.

In this embodiment the curved end portion of the light transmitting element 142 tends to induce the emission of light both laterally as well as directly toward the scalp and thus spread the irradiating light over an area which is larger than in the case of a fiber arrangement wherein the light predominantly is emitted from a tip portion of the fiber(s).

A further aspect of the invention resides in the use of a light source which is used to provide light to the light conducting fibers, to be disposed a handle or the like 160 of a therapeutic coiffuring instrument (such as depicted in FIGS. 3 and 4 for example) which is adapted to be removed from the body in which the fibers are disposed. This ability to separate from the remainder of the therapeutic coiffuring instrument enables the light source 108 to have a dual use and to be used as a flashlight for example during emergency situations or as a source of synthetic daylight which enables a person to dress and or apply make-up and the like while illuminated with the same frequency ranges of light that would occur naturally in sunlight.

In this connection the use of LED which can produce different colors that can be suitably mixed, enables the generation of a wide range of illumination (including “synthetic” daylight and other frequency mixtures which are suitable/selected for healing/therapeutic ends.

For example, depending on the situation, one color of light may be predominately generated, or changed in a predetermined rotation from color to color in order to achieve a predetermined therapeutic coiffuring effect or effects. To enable this selection, the butt of the handle 160, for example, can be provided with rotatable end member 162 which can be rotated to different settings in order to select the appropriate color/intensity. The driver/control circuit can be provided with a processor or the like with a memory which would allow for various treatment programs to be stored and selectively run.

As depicted in FIG. 11, it is within the scope of the present invention to use a mirror of mirrors to reflect light from a light source such as #108 toward the scalp/hair of a user. These mirrors can be used in lieu of or in combination with the light transmitting fibers which are shown in the previously described embodiments. The illumination which is produced will of course produce the same beneficial effects as the embodiments discussed above.

It will be readily seen by one of ordinary skill in the art that embodiments according to the present invention fulfill many of the advantages set forth above. After reading the foregoing specification, one of ordinary skill will be able to affect various changes, substitutions of equivalents and various other aspects of the invention as broadly disclosed herein. It is therefore intended that the protection granted hereon be limited only by the definition contained in the appended claims and equivalents thereof.

What is claimed is:

1. A therapeutic coiffuring instrument comprising:
a light source associated with the therapeutic coiffuring instrument and configured to produce light having a therapeutic effect on scalp and hair; and
elongate members which are configured to contact hair and scalp and to conduct light from the source of light into contact with the hair and scalp.

2. The therapeutic coiffuring instrument as set forth in claim 1, wherein the elongate members are configured to apply low level light directly to the hair and scalp during combing or brushing for therapeutic and cosmetic purposes.

3. The therapeutic coiffuring instrument as set forth in claim 1, wherein the elongate members comprise optical light fibers.

4. The therapeutic coiffuring instrument as set forth in claim 3, wherein the optical light fibers are enclosed in a structure forming part of the therapeutic coiffuring instrument.

5. The therapeutic coiffuring instrument as set forth in claim 3, wherein the optical light fibers are made of plastic or glass.

6. The therapeutic coiffuring instrument as set forth in claim 1, wherein the light source is located in a removable member of the instrument which is detachable from a portion of the instrument which supports the elongate members.

7. The therapeutic coiffuring instrument as set forth in claim 5, wherein the removable member is configured to be hand held and to act as a flashlight.

8. The therapeutic coiffuring instrument as set forth in claim 7, wherein the light source is configured to emit light which is essentially the same as daylight and wherein the member is configured to act as a make-up light.

9. The therapeutic coiffuring instrument as set forth in claim 1, wherein the light source comprises one of a LED (Light Emitting Diodes) and an OLED (Organic Light Emitting Diode).

10. The therapeutic coiffuring instrument as set forth in claim 9, further comprising a battery pack operatively connected with the LED.

11. The therapeutic coiffuring instrument as set forth in claim 10, further comprising a driver circuit which is operatively connected with the battery pack and is configured to control the wavelength of the light produced by the LED.

12. The therapeutic coiffuring instrument as set forth in claim 10, wherein the battery pack is rechargeable using either AC or DC sources.

13. The therapeutic coiffuring instrument as set forth in claim 1, wherein the light source is configured to be connected with a plug-in power supply.

14. The therapeutic coiffuring instrument as set forth in claim 1, wherein the light source comprises a source having a very low power consumption on the order of milliwatts, and a long life in excess of 25,000 hours.

15. The therapeutic coiffuring instrument as set forth in claim 1, wherein the light source is configured so that a wavelength of the light emitted from the light source can be selectively fixed or varied.

16. The therapeutic coiffuring instrument as set forth in claim 1, wherein the light source is configured to double as an emergency light.

17. The therapeutic coiffuring instrument as set forth in claim 1, wherein the light source is configured so that it can be used as a cosmetic application light.

18. The therapeutic coiffuring instrument as set forth in claim 1, having a head portion which is detachable for one of replacement or sanitation.

19. The therapeutic coiffuring instrument as set forth in claim 1, wherein the elongate members are configured to emit light from one or more of their sides and tips.

20. A method of promoting healthy hair, scalp, body and sheen, comprising:
coiffuring hair and scalp with a therapeutic coiffuring instrument comprising:
a light source which is disposed with the therapeutic coiffuring instrument and which is selected to produce therapeutic effects to scalp and hair; and
elongate members which are configured to contact hair and scalp and to conduct light from the source of light into contact with the hair and scalp.
